(19) United States
(12) Patent Application Publication
(10) Pub. No.: US 2020/0297216 A1
(43) Pub. Date: Sep. 24, 2020

(54) PHYSIOLOGICAL MONITORING SYSTEM AND CONTROL METHOD FOR A VITAL-SIGN DETECTION DEVICE

(71) Applicant: MEDIATEK INC., Hsinchu City (TW)

(72) Inventors: Chih-Ming FU, Hsinchu City (TW); Shu-Yu HSU, Hsinchu City (TW); Hung-Chih CHIU, Hsinchu City (TW); Tsan-Jieh CHEN, Hsinchu City (TW)

(21) Appl. No.: 16/742,130

(22) Filed: Jan. 14, 2020

Related U.S. Application Data
(60) Provisional application No. 62/820,911, filed on Mar. 20, 2019.

(51) Int. Cl. A61B 5/0205; A61B 5/11; A61B 5/00

(52) U.S. Cl. CPC A61B 5/0205; A61B 5/1116; A61B 2505/07; A61B 5/7282; A61B 5/1113

(57) ABSTRACT

A physiological monitoring system is provided. The physiological monitoring system includes a vital-sign detection device and a controller. The vital-sign detection device emits visible light during a first period to detect a vital-sign of an object. During the first period, the controller determines whether a first predetermined event occurs. In response to the first predetermined event occurring, the controller controls the vital-sign detection device to emit invisible light during a second period to detect the vital-sign.

### CROSS REFERENCE TO RELATED APPLICATIONS

This application claims the benefit of U.S. Provisional Application No. 62/820,911, filed on Mar. 20, 2019, the contents of which are incorporated herein by reference.

### BACKGROUND OF THE INVENTION

### Field of the Invention

The invention relates to a physiological monitoring system, and more particularly to a physiological monitoring system which can automatically control a photoplethysmography (PPG) sensor to emit at least one of visible light and invisible light.

### Description of the Related Art

With aging societies, more and more burden is placed on hospital resources. Moreover, cardiovascular diseases are increasing, as people age and stress increases for modern day living. Thus, bio-signal self-measurement measurement devices have become an important target for development in the healthcare industry. Through sensing or detecting medically health information, such as electrocardiography (ECG), photoplethysmogram (PPG), heart rate, and blood pressure of patients in bio-signal self-measurement manners, the patients can monitor their own physiology status anytime, to relieve strain on hospital resources and provide needed medical attention to patients. Wearable devices are a hot topic these years. Some wearable devices are capable of tracking medically health information. Among various medically health information, the PPG information is important information which is correlated with the heart rate, oxyhemoglobin saturation (SPO2), blood pressure, sleep stage, occurrence of sleep apnea of the user wearing a wearable device. Generally, a PPG sensor which operates to obtain PPG information comprises a light emitter emitting visible light (such as green light with a better signal-noise ratio). However, when a PPG sensor operates to emit visible light to the user which is ready to sleep or is sleeping, light leakage from the PPG sensor may disadvantageously effect the sleep quality and the body's physiological clock of the user wearing the wearable device especially.

### BRIEF SUMMARY OF THE INVENTION

An exemplary embodiment of a physiological monitoring system is provided. The physiological monitoring system comprises a vital-sign detection device and a controller. The vital-sign detection device emits visible light during a first period to detect a vital-sign of an object. During the first period, the controller determines whether a first predetermined event occurs. In response to the first predetermined event occurring, the controller controls the vital-sign detection device to emit invisible light during a second period to detect the vital-sign.

An exemplary embodiment of a control method for a vital-sign detection device. The control method comprises the steps of controlling the vital-sign detection device to emit visible light during a first period to detect a vital-sign of an object; during the first period, determining whether a first predetermined event occurs; and in response to the first predetermined event occurring, controlling the vital-sign detection device to emit invisible light during a second period to detect the vital-sign.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 shows one exemplary embodiment of a physiological monitoring system;

FIGS. 2A and 2B are schematic diagrams showing a vital-sign detection device according to exemplary embodiments;

FIG. 3 shows an exemplary embodiment of a control method for the vital-sign detection device;

FIGS. 4A and 4B are a schematic diagrams showing emitting states of visible light and invisible light according to exemplary embodiments;

FIG. 5 is flow chart showing details of the step S31 of FIG. 3 according to an exemplary embodiment;

FIGS. 6A and 6B are schematic diagrams showing variation in motion of a user detected by a motion detector according to an exemplary embodiment;

FIG. 7 is a schematic diagram showing variation in a heart rate of a user detected by a heart-rate detector according to an exemplary embodiment;

FIG. 8 is flow chart showing details of the step S34 of FIG. 3 according to an exemplary embodiment; and

FIG. 9 is a schematic diagram showing various apparatus in the physiological monitoring system of FIG. 1 according to an embodiment.

### DETAILED DESCRIPTION OF THE INVENTION

The following description is of the best-contemplated model of carrying out the invention. This description is made for the purpose of illustrating the general principles of the invention and should not be taken in a limiting sense. The scope of the invention is best determined by reference to the appended claims.

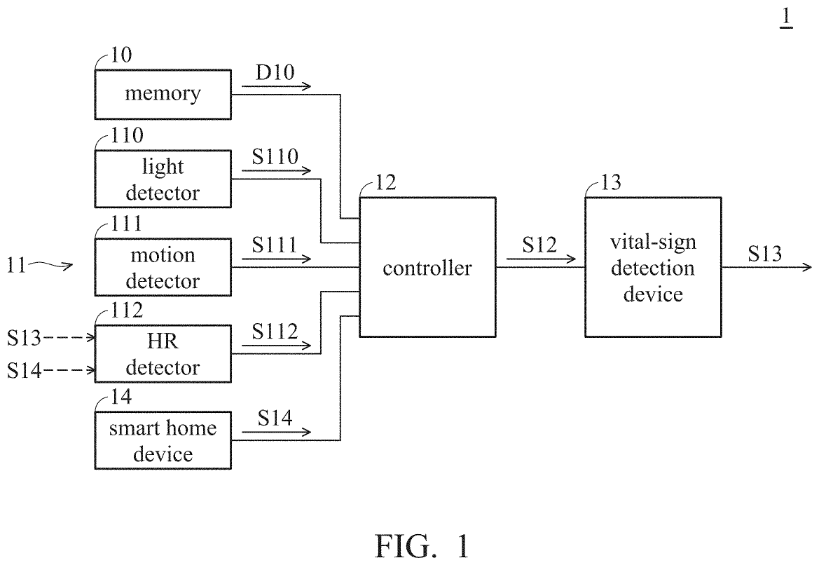

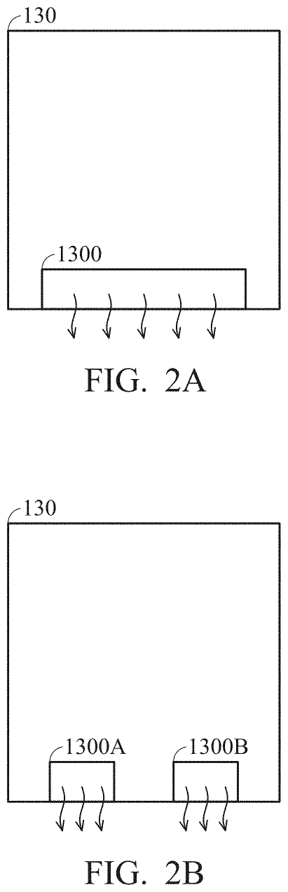

FIG. 1 shows one exemplary embodiment of a physiological monitoring system. As shown in FIG. 1, a physiological monitoring system 1 is provided. In the embodiment, the physiological monitoring system 1 operates to monitor at least one vital-sign of an object, such as a user, to generate a vital-sign signal. In an embodiment, the monitored vital-sign is the photoplethysmography (PPG) of the user using or wearing the physiological monitoring system 1. The physiological monitoring system 1 can automatically control a photoplethysmography (PPG) sensor to emit invisible light before the user falls asleep or during the period when the user is sleeping and then control the PPG sensor to emit visible light in response to the user awaking from the sleep. As shown in FIG. 1, the physiological monitoring system 1 comprises a memory 10, a plurality of detectors 11, a controller 12, and a vital-sign detection device 13. In another embodiment, the vital-sign detection system 1 further comprises a smart home device 14 which can communicate with electronic products/devices in the user's place, such as smart lamps. The memory 10 may store preset sleep time which was input previously by the user or obtained from historical sleep time calculated by the controller 12 (the detailed description will be shown later). According to an embodiment, the plurality of detectors 11 comprises a light detector 110, a motion detector 111, and a heart-rate (HR) detector 112. The vital-sign detection device 13 may emit visible light and invisible light for sensing pulses of a blood vessel of the user to generate a vital-sign signal S13. According to the embodiment, the visible light can be the light whose wavelength is in a range from about 380 nm to about 760 nm, and the invisible light can be the light whose wavelength is less than about 380 nm or larger than about 760 nm. For example, in an embodiment, the visible light is green light, and the invisible light is infrared. As shown in FIG. 2A, the vital-sign detection device 13 comprises a PPG sensor 130, and the PPG sensor 130 comprises one light emitter 1300 which can emit light having an adjustable wavelength. The light emitter 1300 is controlled by the controller 12 to adjust the adjustable wavelength of the light, so that the light emitter 1300 emits visible light or invisible light through the adjustment of the adjustable wavelength. The position of the light emitter 1300 shown in FIG. 2A is an example for illustrating the light emitting from the PPG sensor 130, and the real position of the light emitter 1300 in the PPG 130 is determined according to the system design. In another embodiment, as shown in FIG. 2B, the PPG sensor 130 comprises a light emitter 1300A which is configured to emit visible light and a light emitter 1300B which is configured to emit invisible light. Since the light emitters 1300A and 1300B are independence from each other, the controller 12 can control the vital-sign detection device 130 to emit at least one of the visible light from the light emitter 1300A and the invisible light from the light emitter 1300B at a time. Thus, the period when the light emitter 1300A emits the visible light does not overlap the period when the light emitter 1300B emits the invisible light or the period when the light emitter 1300A emits the visible light partially overlaps the period when the light emitter 1300B emits the invisible light. The positions of the light emitters 1300A and 1300B shown in FIG. 2B are an example for illustrating the light emitting from the PPG sensor 130, and the real positions of the light emitters 1300A and 1300B in the PPG 130 is determined according to the system design. The controller 12 generates a control signal S12 and controls the vital-sign detection device 13 through the control signal S12 according to the signals/data from the memory 10, the plurality of detectors 11, and/or the smart home device 13.

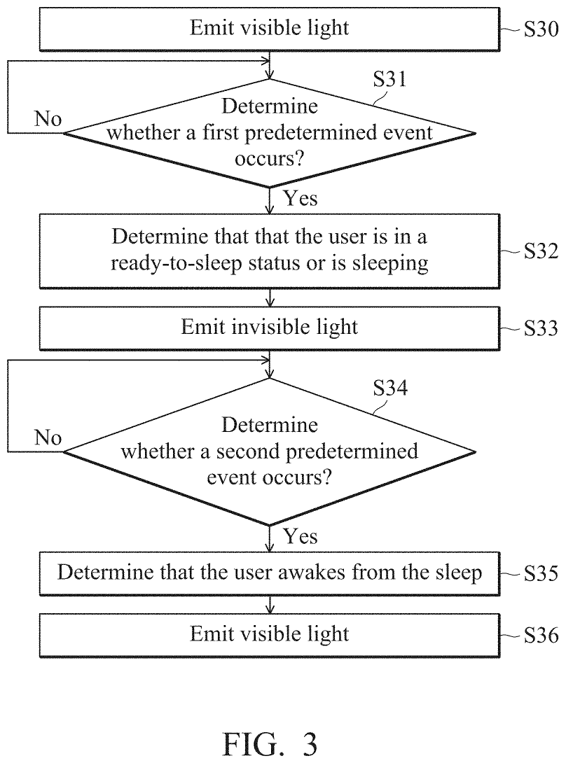

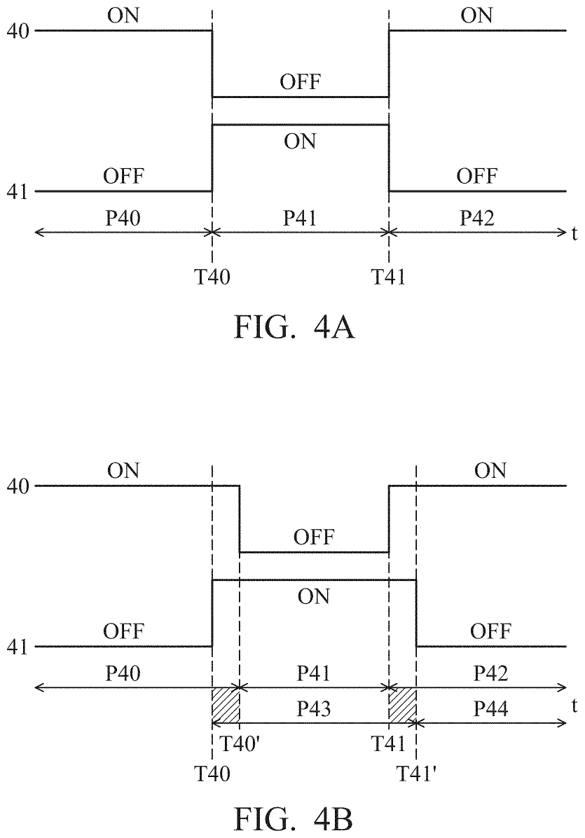

FIG. 3 shows an exemplary embodiment of a control method for the vital-sign detection device 13. Referring to FIG. 3, the vital-sign detection device 13 initially emits the visible light from the PPG sensor 130 (Step S30). Referring to FIGS. 4A and 4B, the labels 40 and 41 represents the emitting states of the visible light and the invisible light respectively, wherein “ON” indicates that the corresponding light is being emitted by the PPG sensor 130, while “OFF” indicates that the light is not being emitted by the PPG sensor 130. In FIGS. 4A and 4B, the vital-sign detection device 13 initially emits the visible light during the period P40 (40: ON). Referring to FIG. 3 again, the controller 12 then determines whether a first predetermined event occurs during the period P40 when the vital-sign detection device 13 emits the visible light (Step S31). In the embodiment, the first predetermined event indicates that the user is in a ready-to-sleep status which occurs before the user falls asleep (such as, a state in which the user is in a lying posture or still for a while) or the user is sleeping (such as, the user breathes regularly). If the controller 12 determines that the first predetermined event does not occur, the step S31 is performed repeatedly. Once the controller 12 determines that the first predetermined event occurs, the controller 12 determines that the user is in the ready-to-sleep status or is sleeping (Step S32) and controls the vital-sign detection device 13 to emit the invisible light (41: ON) through the control signal S12 (Step S33). In one embodiment, referring to FIG. 4A, when the controller 12 determines that the first predetermined event occurs at the time point T40, the controller 12 controls the PPG sensor 130 to stop emitting the visible light (40: OFF) and emit the invisible light (41: ON) at the same time point T40. Thus, during the period P41 starting from the time point T40, the PPG sensor 130 emits the invisible light (41: ON), but does not emit the visible light (40: OFF). In this embodiment, the period P41 when the invisible light is emitted (41: ON) does not overlap the period P40 when the visible light is emitted (40: ON). In another embodiment, referring to FIG. 4B, when the controller 12 determines that the first predetermined event occurs at the time point T40, the controller 12 controls the PPG sensor 130 to emit the invisible light (41: ON) at the time point T40. Then, at the time point T40′ occurring after the time point T40, the controller 12 controls the PPG sensor 130 to stop emitting the visible light (40: OFF). Thus, the PPG sensor 130 emits the invisible light (41: ON) during the period P43 starting from the time point T40, and the PPG sensor 130 does not emit the visible light (40: OFF) during the period P43 starting from the time point T40′. In this embodiment, the period P43 when the invisible light is emitted (41: ON) partially overlaps the period P40 when the visible light is emitted (40: ON) as shown by the oblique lines in FIG. 4B, wherein the period P40 ends during the period P43.

In an embodiment, the controller 12 defines each time point T40 when the first predetermined event occurs as a sleep time. When the controller 12 obtains at least one time point T40, the controller 12 calculates historical sleep time according to the least one time points T40 by using statistical manners and provides a signal which contains information about the historical sleep time to the memory 10 for updating the preset sleep time.

During the period P41 (FIG. 4A)/P43 (FIG. 4B) when the PPG sensor 130 emits the invisible light, the controller 12 determines whether a second predetermined event occurs (step S34). In the embodiment, the second predetermined event indicates that the user awakes from the sleep. If the controller 12 determines that the second predetermined event does not occur, the step S34 is performed repeatedly, and, at this time, the PPG sensor 130 continuously emits only the invisible light. Once the controller 12 determines that the second predetermined event occurs, the controller 12 determines that the user awakes from the sleep (Step S35) and controls the vital-sign detection device 13 to emit the visible light (40: ON) through the control signal S12 (Step S36). In one embodiment, referring to FIG. 4A, when the controller 12 determines that the second predetermined event occurs at the time point T41, the controller 12 controls the PPG sensor 130 to stop emitting the invisible light (41: OFF) and emit the visible light (40: ON) at the same time point T41. Thus, during the period P42 starting from the time point T41, the PPG sensor 130 emits the visible light (40: ON), but does not emit the invisible light (41: OFF). In this embodiment, the period P42 when the visible light is emitted (40: ON) does not overlap the period P41 when the invisible light is emitted (41: ON). In another embodiment, referring to FIG. 4B, when the controller 12 determines that the first predetermined event occurs at the time point T41, the controller 12 controls the PPG sensor 130 to emit the visible light (40: ON) at the time point T41. Then, at the time point T41′ occurring after the time point T41, the controller 12 controls the PPG sensor 130 to stop emitting the invisible light (41: OFF). Thus, the PPG sensor 130 emits the visible light (40: ON) during the period P42 starting from the time point T41, and the PPG sensor 130 does not emit the invisible light (41: OFF) during the period P44 starting from the time point T41′. In this embodiment, the period P42 when the visible light is emitted (40: ON) partially overlaps the period P43 when the invisible light is emitted (41: ON) as shown by the oblique lines in FIG. 4B, wherein the period P43 ends during the period P42.

In the above, the emitting states of the visible light and the invisible light shown in FIG. 4A can be achieved by using the PPG sensor 130 of FIG. 2A or the PPG sensor 130 of FIG. 2B, while the emitting states of the visible light and the invisible light shown in FIG. 4A can be achieved by using the PPG sensor 130 of FIG. 2B.

According to the embodiment, the physiological monitoring system 1 can automatically control the PPG sensor 130 to stop emitting the visible light and begin emitting the invisible light before the user falls asleep or during the period when the user is sleeping. The physiological monitoring system 1 can also automatically control the PPG sensor 130 to begin emitting visible light in response to the user awaking from the sleep. Thus, during the period when the user is sleeping, the visible light cannot be sensed by the eyes of the user, thereby avoiding affecting the sleep quality and the body's physiological clock of the user by the light leakage from the PPG sensor 130.

In the embodiment, for determining whether the first predetermined event occurs in the step S31, the controller 12 sets a plurality of first conditions and determines whether each of the plurality of first conditions is met. In the embodiment, the controller 12 sets four first conditions. In the cases where some first conditions are met, the controller 12 determines whether the number (N) of the first conditions which are met is larger than a first threshold X. If the controller 12 determines that the number of the first conditions which are met is larger than the first threshold X, the controller 12 determines that the first predetermined event occurs. According to the embodiment, the first threshold (X) is set to be 70%˜80% of the total number of first conditions. For example, in the cases where there are four first conditions, the first threshold is set as 3 (X=3). In the following paragraphs, how the controller 12 determines whether the first predetermined event occurs will be described, that is, the detail of the step S31 will be described.

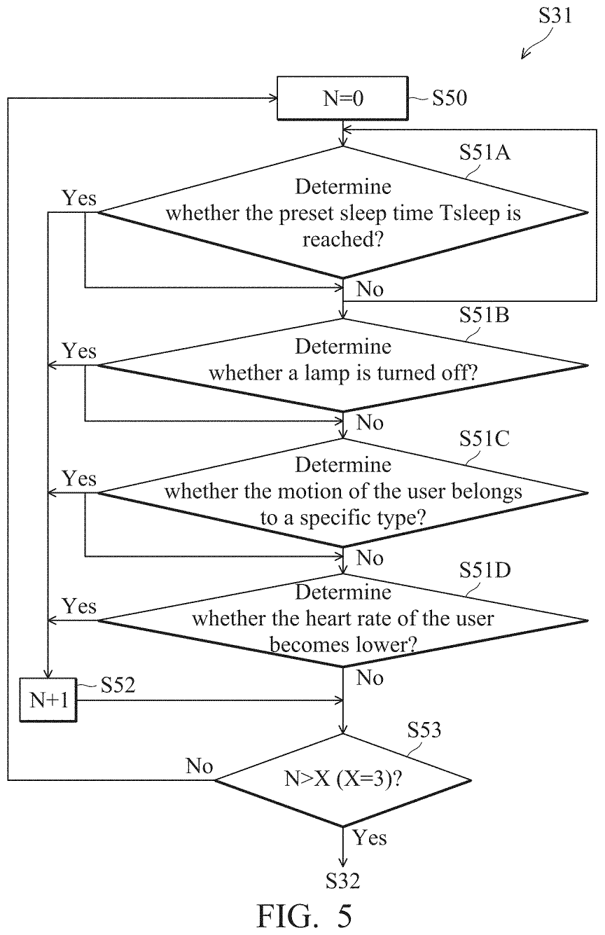

In the embodiment, the controller 12 generates a counting value through a counting operation of an internal counter. Referring to FIG. 5, the controller 12 resets the counting value N to “0” (Step S50: N=0). Then, the controller 12 accesses the memory 10 to read the data D10 containing the preset sleep time Tsleep and determines whether the preset sleep time Tsleep is reached (Step S51A). In FIG. 5, the step S51A is represented as “determine whether Tsleep is reached?” Once the preset sleep time Tsleep is reached, the controller 12 determines that one of the plurality of first conditions is met and increases the counting value N by “1” (Step S52: N+1). If the preset sleep time Tsleep is not reached yet, the controller 12 continuously determines whether the preset sleep time Tsleep is reached (Step S51A), and the flow proceeds to the step S51B.

Referring to FIG. 5, in the step S51B, the controller 12 determines whether a lamp near the vital-sign detection device 13 is turned off. If the controller 12 determines that lamp near the vital-sign detection device 13 is turned off, the controller 12 determines that one of the plurality of first conditions is met and increases the counting value N by “1” (Step S52: N+1). Referring to FIG. 1, the light detector 110 detects ambient light of the vital-sign detection device 13 and generates a light-detection signal S110 according to the detected ambient light. The controller 12 receives the light-detection signal S110 and analyzes the light-detection signal S111 to obtain the intensity of the ambient light which can indicate the on/off state of the lamp. In an embodiment, whether the lamp near the vital-sign detection device 13 is turned off is determined according to the intensity of the ambient light. According to an embodiment, the intensity of the ambient light is obtained by the following algorithm. First, the controller 12 calculates the mean value of the luminous flux (lux) of the detected ambient light in 1 minute, wherein the calculated mean value serves as the above intensity of the ambient light. The controller 12 determines whether the calculated mean is less than a first predetermined threshold (such as 5 lm) for more than a predetermined period (such as, 5 minutes) and further determines whether the calculated mean is larger than a second predetermined threshold (such as 50 lm) for more than the predetermined period (5 minutes). If the calculated mean is less than 5 lm for more than 5 minutes, the controller 12 determines that the lamp near the vital-sign detection device 13 is turned off, which can represent that the user is in the ready-to-sleep status or is sleeping. If the calculated mean is larger than 50 lm for more than 5 minutes, the controller 12 determines that the lamp near the vital-sign detection device 13 is not turned off (that is, the lamp is turned on), which can represent that the user is not in the ready-to-sleep status and not sleeping.

In another embodiment, in the cases where the lamp near the vital-sign detection device 13 is a smart lamp, the smart home device 14 can communicate with the smart lamp to control its on/off state and then generate an indication signal S14 according to the current on/off state of the smart lamp. The controller 12 receives the indication signal S14 and determines whether the smart lamp is turned off according to the indication signal S14.

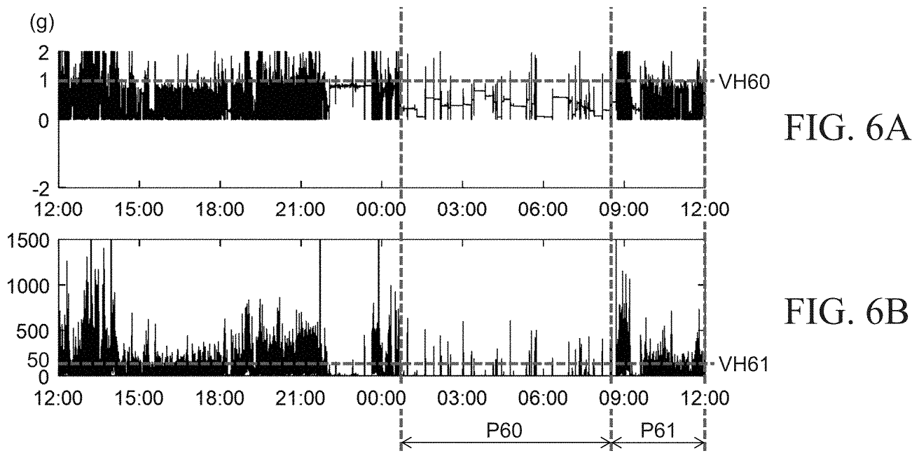

Referring to FIG. 5, after the determination at the step S51B is done, the controller 12 determines whether the motion of the user belongs to a specific type (Step S51C). In the embodiment, the specific type indicates that the user is in a lying posture, is still for a while, or breathes regularly which can be represented by regular moving of the thoracic cavity of the user. For example, the specific type indicates that the user is in a lying posture and/or still for a while. Referring to FIG. 1, the motion detector 111 detects the motion of the user and generates a motion signal S111 according to the detected motion. The motion sensor 112 provides the motion signal S111 to the controller 12. In the embodiment, the motion detector 111 may comprise at least one device which can provide motion information of a specific object detected or monitored by the least one device, such as at least one of an accelerometer, a gyroscope, and a camera. The motion information indicates whether the user is in a laying posture or still for a while or breathes regularly. In the following, an embodiment where the motion detector 111 detects the motion of the user by a gyroscope will be described. Based on a general operation of a gyroscope, the signal generated by the gyroscope contains three components: X-axis component, Y-axis component, and Z-axis component. Accordingly, the motion signal S111 generated by the motion sensor 111 contains an X-axis component, a Y-axis component, and an Z-axis component for the gyroscope. Referring to FIG. 6A, in the cases where the user is lying on the bed and sleeping during the period P60, the value of the X-axis is less during the period P60, for example, the value of the X-axis component is less than 1 g (9.8 m/s²). Thus, in the embodiment, the controller 12 determines whether the value of the X-axis component contained in the motion signal S111 is less than a predetermined threshold VH60, such as 1 g (9.8 m/s²), thereby determining whether the user is in a lying posture. If the value of the X-axis component is less than the predetermined threshold VH60, the controller 12 determines that the user is in the lying posture (that is, the motion of the user belongs to the specific type) and determines that one of the plurality of first conditions is met. Then, the controller 11 increases the counting value N by “1” (Step S52: N+1).

Referring to FIG. 6B, during the period P60 when the user is lying on the bed and sleeping, the activity of the user is less. Thus, in another embodiment, the controller 12 receives the motion signal S111 and analyzes it to obtain the activity of the user. The controller 12 determines whether the obtained activity of the user is less than a predetermined threshold VH61 (such as 50) for more than a predetermined period (for example, 5 minutes), thereby determining whether the user is still for a while. If the obtained activity of the user is less than 50 for 5 minutes, the controller 12 determines that the user is still for a while (that is, the motion of the user belongs to the specific type) and determines that one of the plurality of first conditions is met. Then, the controller 12 increases the counting value N by “1” (Step S52: N+1).

According to an embodiment, the activity of the user is obtained by the following algorithm. The values of the X-axis component, Y-axis component, and Z-axis component of the gyroscope are represented by x, y, and z respectively. After receiving the motion signal S111, the controller 12 calculates the square root of the sum of the square of x, the square of y, and the square of z to obtain an original activity value Activity_original (Activity_original=Sqet(x²+y²+z²). Then, the controller 12 performs high pass filtering (HPF) on the original activity value Activity_original to obtain a filtered activity value Activity_filtered (Activity_filtered=HPF(Activity_original)). The controller 12 calculates the mean value of the filtered activity values Activity_filtered which are obtained every 10 minutes to obtain a mean activity MA_Activity (MA_Activayr=mean (Activity_filtered in 10 minutes)), wherein the mean activate MA_Activity serves as the above the activity of the user. Then, the controller 12 determines whether the mean activate MA_Activity is less than 50 for more than 5 minutes ((MA_Activity<50) over 5 minutes). If the mean activate MA_Activity is less than 50 for more than 5 minutes, the controller 12 determines that the user is still for a while and determines that one of the plurality of first conditions is met.

In another embodiment, the controller 12 may determine whether the motion of the user belongs to the specific type by determining whether the user is in a lying posture and determining whether the user is still for a while. If the controller 12 determines that the user is in the lying posture, that the user is still for a while, or that the user is in the lying posture and sill for a while, the controller 12 determines that the motion of the user belongs to the specific type.

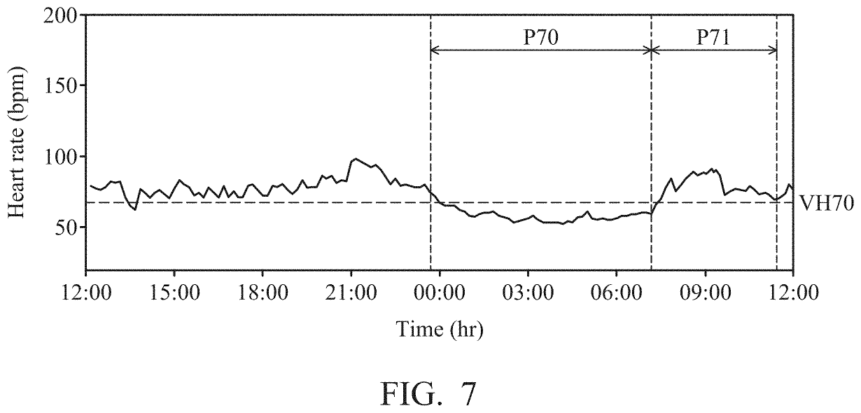

Referring to FIG. 5, after the determination at the step S51C is done, the controller 12 determines whether the heart rate of the user becomes lower (Step S51D). Referring to FIG. 1, the heart-rate detector 112 may receive the vital-sign signal S13 from the vital-sign detection device 13 and/or an ECG signal S14 from an ECG monitor and obtain the heart rate of the user according to the vital-sign detection device 13 and/or the ECG signal S14. How to obtain a heart rate of a user contacting a PPG sensor or an ECG monitor is well known by the one skilled in the art, thus, the related description is omitted here. The heart-rate detector 112 generates a detection signal S112 according to the obtained heart rate. Referring to FIG. 7, in the cases where the user is sleeping during the period P70, the heart rate value of the user is less during the period P70, for example, the average of the heart rate is 56.7 bpm. Thus, in the embodiment, the controller 12 receives the detection signal S112, obtains the heart rate of the user from the detection signal S112, and determines whether the heart rate of the user becomes lower than a predetermined threshold VH70 for more than a predetermined period, thereby determining whether the user is sleeping. If the heart rate of the user becomes lower than the predetermined threshold VH70 for more than the predetermined period, the controller 12 determines that the user is sleeping and determines that one of the plurality of first conditions is met. Then, the controller 11 increases the counting value N by “1” (Step S52: N+1).

After the steps S51A˜S51D are done, the counting value N represents the number of first conditions are met. The controller 12 determines whether the counting value N is larger than the first threshold X (Step S53: N>X (X=3)?). If the controller 12 determines that the counting value N is larger than the first threshold X, the controller 12 determines that the first predetermined event occurs, and the flow proceeds to the step S32 of FIG. 3. If the controller 12 determines that the counting value N is not larger than the first threshold X, the controller 12 determines that the first predetermined event does not occur, and the step S31 is performed repeatedly.

In the embodiment, for determining whether the second predetermined event occurs in the step S34, the controller 12 sets a plurality of second conditions and determines whether each of the plurality of second conditions is met. In the embodiment, the controller 12 sets three second conditions. In the cases where some second conditions are met, the controller 12 determines whether the number (M) of the second conditions which are met is larger than a second threshold Y. If the controller 12 determines that the number (M) of the second conditions which are met is larger than the second threshold Y, the controller 12 determines that the second predetermined event occurs. According to the embodiment, the second threshold (Y) is set to be 65%˜80% of the total number of second conditions. For example, in the cases where there are three second conditions, the second threshold is set as 2 (Y=2). In the following paragraphs, how the controller 12 determines whether the second predetermined event occurs will be described, that is, the detail of the step S34 will be described.

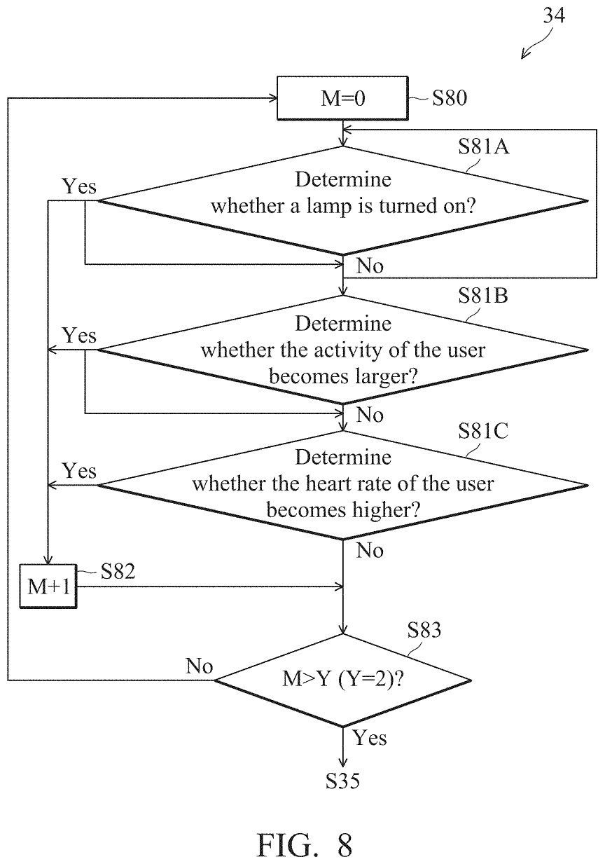

In the embodiment, the controller 12 generates a counting value M through a counting operation of another internal counter. Referring to FIG. 8, the controller 12 resets the counting value M to “0” (Step S80: M=0). Then, the controller 12 determines whether a lamp near the vital-sign detection device 13 is turned on (Step S81A). If the controller 12 determines that lamp near the vital-sign detection device 13 is turned on, the controller 12 determines that one of the plurality of second conditions is met and then increases the counting value M by “1” (Step S82: M+1). As described above, the controller 12 determines whether the intensity of the ambient light (the mean value of the luminous flux (lux) of the detected ambient light in 1 minute) is less than 5 lm for more than a predetermined period (such as, 5 minutes) and further determines whether the intensity of the ambient light is larger than 50 lm for more than 5 minutes. If the calculated mean is larger than 50 lm for more than 5 minutes, the controller 12 determines that the lamp near the vital-sign detection device 13 is turned on, which can represent that the user awakes from the sleeping. If the calculated mean is less than 5 lm for more than 5 minutes, the controller 12 determines that the lamp near the vital-sign detection device 13 is turned off, which can represent that the user is still sleeping.

Referring to FIG. 6B, when the user awakes from the sleep during the period P61, the activity of the user becomes larger. Thus, as shown in FIG. 8, after the determination at the step S81A is done, the controller 12 determines whether the activity of the user becomes larger (Step S81B), thereby determining whether the user awakes from the sleep. In the embodiment, the controller 12 determines whether the activity of the user becomes larger than the predetermined threshold VH61 for more than a predetermined period (for example, 5 minutes). If the obtained activity of the user is larger than the predetermined threshold VH61 for 5 minutes, the controller 12 determines that the user awakes from the sleep and determines that one of the plurality of second conditions is met. Then, the controller 12 increases the counting value M by “1” (Step S82: M+1). If the obtained activity of the user does not become larger than the predetermined threshold VH61 for 5 minutes, the controller 12 determines that the user is still sleeping.

Referring to FIG. 7, when the user awakes from the sleep during the period P71, the heart rate value of the user becomes higher, for example, the average of the heart rate is 76.2 bpm. Thus, as shown in FIG. 8, after the determination at the step S81B is done, in the embodiment, the controller 12 determines whether the heart rate of the user becomes higher (Step S81C), thereby determining whether the user awakes from the sleep. According to an embodiment, in the Step S81C, the controller 12 determines whether the heart rate of the user becomes higher than the predetermined threshold VH70 for more than a predetermined period. If the heart rate of the user becomes higher than the predetermined threshold VH70 for more than the predetermined period, the controller 12 determines that the user awakes from the sleep and determines that one of the plurality of second conditions is met. Then, the controller 12 increases the counting value M by “1” (Step S82: M+1). If the heart rate of the user does not become higher than the predetermined threshold VH70 for more than the predetermined period, the controller 12 determines the controller 12 determines that the user is still sleeping.

After the steps S81A˜S81C are done, the counting value M represents the number of second conditions are met. The controller 12 determines whether the counting value M is larger than the second threshold Y (Step S83: M>Y (Y=2)?). If the controller 12 determines that the counting value M is larger than the first threshold Y, the controller 12 determines that the second predetermined event occurs, and the flow proceeds to the step S35 of FIG. 3. If the controller 12 determines that the counting value M is not larger than the first threshold Y, the controller 12 determines that the second predetermined event does not occur, and the step S34 is performed repeatedly.

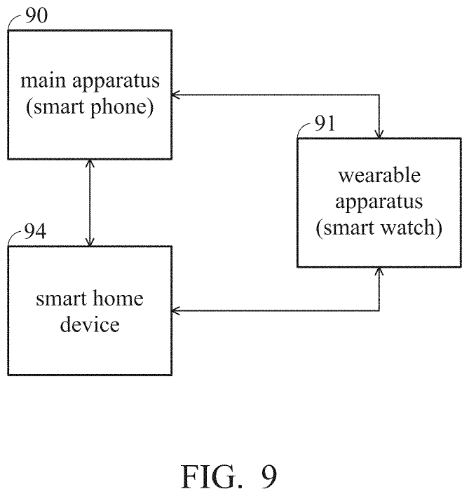

In an embodiment, the physiological monitoring system 11 comprises several apparatus, and the devices/elements shown in FIG. 1 can be disposed on these apparatus. Referring to FIG. 9, in addition to the smart home device 14, the vital-sign detection system 1 further comprises two apparatus: a main apparatus 90 and a wearable apparatus 91. For example, the main apparatus 90 can be a smart phone, while the wearable apparatus 91 is a smart watch worn by the user. According to the above description, the motion detector 111 and the vital-sign detection device 13 are disposed on the smart watch 91 based on their operations and functions. In an embodiment, the memory 10 can be disposed on the smart phone 90 or the smart watch 91, and the data D10 in the memory 10 related to the sleep time is input by the user previously or obtained from historical sleep time detected by the controller 12. In an embodiment, the light detector 110 may be disposed on the smart phone 90 or the smart switch 91. In another embodiment, the light detector 110 may be disposed on the smart home device 14 in the cases where the smart home device 14 is on the location where the user sleeps, such as, the user's bedroom. The controller 12 is disposed on smart phone 90 or smart watch 91. In other embodiment, the controller 12 can be implemented by the processor of the smart phone 90 or smart watch 91.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A physiological monitoring system comprising:
a vital-sign detection device emitting visible light during a first period to detect a vital-sign of an object; and
a controller, during the first period, determining whether a first predetermined event occurs,
wherein in response to the first predetermined event occurring, the controller controls the vital-sign detection device to emit invisible light during a second period to detect the vital-sign.

2. The physiological monitoring system as claimed in claim 1, wherein the first period is followed by the second period.

3. The physiological monitoring system as claimed in claim 1, wherein the second period partially overlaps the first period.

4. The physiological monitoring system as claimed in claim 1,
wherein during the second period, the controller determines whether a second predetermined event occurs; and
wherein in response to the second predetermined event occurring, the controller controls the vital-sign detection device to emit the visible light during a third period to detect the vital-sign.

5. The physiological monitoring system as claimed in claim 4, wherein the second period is followed by the third period.

6. The physiological monitoring system as claimed in claim 4, wherein the third period partially overlaps the second period.

7. The physiological monitoring system as claimed in claim 1, wherein the vital-sign detection device comprises:
a photoplethysmography (PPG) sensor comprising a light emitter which emits light having an adjustable wavelength,
wherein the controller controls the light emitter to adjust the adjustable wavelength to emit the light as the visible light during the first period and emit the light as the invisible light during the second period.

8. The physiological monitoring system as claimed in claim 7, wherein the second period does not overlap the first period.

9. The physiological monitoring system as claimed in claim 1, wherein the vital-sign detection device comprises:
a photoplethysmography (PPG) sensor comprising a first light emitter emitting the visible light and further comprising a second light emitter emitting the invisible light;
wherein the controller controls the vital-sign detection device to emit at least one of the visible light from the first light emitter and the invisible light from the second light emitter at a time.

10. The physiological monitoring system as claimed in claim 9, wherein in response to the first predetermined event occurring, the controller controls the vital-sign detection device to stop emitting the visible light from the first light emitter and emit the invisible light from the second light emitter during the second period.

11. The physiological monitoring system as claimed in claim 9, wherein in response to the first predetermined event occurring, the first period when the vital-sign detection device emits the visible light from the first light emitter ends during the second period when the vital-sign detection device emits the invisible light from the second light emitter.

12. The physiological monitoring system as claimed in claim 1,
wherein the controller sets a plurality of conditions and determines whether each of the plurality of conditions is met, and
wherein if the number of conditions which are met is larger than a threshold, the controller determines that the predetermined event occurs.

13. The physiological monitoring system as claimed in claim 12, further comprising:
a motion detector detecting motion of the object and generating a motion signal according to the detected motion,
wherein the controller determines whether the motion of the object belongs to a specific type according to the motion signal,
wherein in response to the controller determining that the motion of the object belongs to the specific type, the controller determines that one of the plurality of conditions is met.

14. The physiological monitoring system as claimed in claim 13, wherein the specific type indicates that the object is in a lying posture or the object is still for a while.

15. The physiological monitoring system as claimed in claim 13, wherein the specific type indicates that the object breathes regularly.

16. The physiological monitoring system as claimed in claim 13, wherein the motion of the object belonging to the specific type occurs when the object is sleeping.

17. The physiological monitoring system as claimed in claim 12, further comprising:
a light detector detecting ambient light of the vital-sign detection device and generating a light-detection signal according to the detected ambient light,
wherein the controller determines whether a lamp near the vital-sign detection device is turned off according to the light-detection signal,
wherein in response to the controller determining that the lamp is turned off, the controller determines that one of the plurality of conditions is met.

18. The physiological monitoring system as claimed in claim 12, further comprising:
a smart home device controlling an on/off state of a smart lamp near the vital-sign detection device and generating an indication signal according to the current on/off state,
wherein the controller determines whether the smart lamp near the vital-sign detection device is turned off according to the indication signal,
wherein in response to the controller determining that the smart lamp is turned off, the controller determines that one of the plurality of conditions is met.

19. The physiological monitoring system as claimed in claim 12, wherein the vital-sign detection device comprises:
a heart-rate detector detecting a heart rate of the object and generating a detection signal according to the detected heart rate,
wherein the controller receives the detection signal and determines whether the detected heart rate is lower than a predetermined threshold for more than a predetermined period,
wherein in response to the controller determining that the detected heart rate is lower than the predetermined threshold for more than the predetermined period, the controller determines that one of the plurality of conditions is met.

20. The physiological monitoring system as claimed in claim 12, further comprising:
a memory storing preset sleep time of the object,
wherein the controller determines whether the preset sleep time is reached,
wherein in response to the controller determining that the preset sleep time is reached, the controller determines that one of the plurality of conditions is met the first predetermined event occurs.

21. A control method for a vital-sign detection device comprising:
controlling the vital-sign detection device to emit visible light during a first period to detect a vital-sign of an object;
during the first period, determining whether a first predetermined event occurs; and
in response to the first predetermined event occurring, controlling the vital-sign detection device to emit invisible light during a second period to detect the vital-sign.

22. The control method as claimed in claim 21, wherein the first period is followed by the second period.

23. The control method as claimed in claim 21, wherein the second period partially overlaps the first period.

24. The control method as claimed in claim 21, further comprising:
during the second period, determining whether a second predetermined event occurs; and
in response to the second predetermined event occurring, emit the visible light during a third period to detect the vital-sign.

25. The control method as claimed in claim 24, wherein the second period is followed by the third period.

26. The control method as claimed in claim 24, wherein the third period partially overlaps the second period.

27. The control method as claimed in claim 21, wherein determining whether a first predetermined event occurs comprises:
setting a plurality of conditions;
determines whether each of the plurality of conditions is met;
counting the number of conditions which are met; and
determining whether the number of conditions which are met is larger than a threshold; and
in response to the number of conditions which are met being larger than the threshold, determining that the first predetermined event occurs.

28. The control method as claimed in claim 27, wherein determining whether each of the plurality of conditions is met comprises:
detecting motion of the object;
determining whether the motion of the object belongs to a specific type according to the detected motion;
in response to the controller determining that the motion of the object belongs to the specific type, determining that one of the plurality of conditions is met.

29. The control method as claimed in claim 28, wherein the specific type indicates that the object is in a lying posture, the object is still for a while, or the object breathes regularly.

30. The control method as claimed in claim 27, wherein determining whether each of the plurality of conditions is met comprises:
detecting intensity of ambient light of the vital-sign detection device;
determining whether the detected intensity of the ambient light is lower than a predetermined threshold; and
in response to determining that the detected intensity of the ambient light is lower than the predetermined threshold, the controller determines that one of the plurality of conditions is met.

31. The control method as claimed in claim 27, wherein determining whether each of the plurality of conditions is met comprises:
detecting a heart rate of the object;
determining whether the detected heart rate is lower than a predetermined threshold for more than a predetermined period,
in response to determining that the detected heart rate is lower than the predetermined threshold for more than the predetermined period, determining that one of the plurality of conditions is met.

32. The control method as claimed in claim 27, further comprising:
setting sleep time of the object;
determining whether the preset sleep time is reached; and
in response to determining that the preset sleep time is reached, determining that one of the plurality of conditions is met.
